(19) United States
(12) Patent Application Publication
(10) Pub. No.: US 2025/0044278 A1
(43) Pub. Date: Feb. 6, 2025

(54) OPTICAL DENSITY MEASUREMENT AND TESTING DEVICE

(71) Applicants: Russell Garry Latimer, Brentwood, TN (US); Willard Watson Young, III, Brentwood, TN (US); David John Vigerust, Brentwood, TN (US)

(72) Inventors: Russell Garry Latimer, Brentwood, TN (US); Willard Watson Young, III, Brentwood, TN (US); David John Vigerust, Brentwood, TN (US)

(21) Appl. No.: 18/227,982

(22) Filed: Jul. 31, 2023

Related U.S. Application Data
(63) Continuation-in-part of application No. 17/692,493, filed on Mar. 11, 2022, now abandoned.
Application No. 17/692,493 is a continuation of application No. 16/435,445, filed on Jun. 7, 2019, now abandoned.

(51) Int. Cl. G01N 33/50; G01N 15/06

(52) U.S. Cl. CPC G01N 33/5011; G01N 15/06; G01N 15/01; G01N 15/075; G16H 20/10; G16H 50/20

(57) ABSTRACT

The embodiments disclose an optical density measurement and testing system including a purification device configured to detect and isolate live cancer cells of a patient microbiology sample, a drug addition device configured to infuse a drug treatment to the live cancer cells, an optical density device configured to capture images of the microbiology sample, an optical microplate spectrophotometric reader coupled to the optical density device configured to measure the density of living cells after infusion of the at least one drug treatment, a computer coupled to the optical microplate spectrophotometric reader configured to determine measured rates of death over a period of time based on the interval population growth data, and an optical density application operating on the computer configured to use the measured rates of death and known predetermined genetic markers, resistances and allergies of the patient associated with the drug to generate clinician treatment recommendations for the patient.

### BACKGROUND

Testing of products and materials is performed in most industries and professions. Measurements are the key to providing the results of the testing. What size, shape, temperature, viscosity, and other factors are needed to determine the outcome for testing results and verify whether the products and materials meet the initial design criteria. To perform accurate measurements a myriad of devices and systems are needed based on the nature of the testing and physical environment. Many measurement devices and systems are unknown for particular testing which could be a good match to the testing and provide more accurate results than previously understood.

### BRIEF SUMMARY OF THE INVENTION

The embodiments disclose an optical density measurement and testing system including a purification device configured to detect and isolate live cancer cells of a patient microbiology sample. A drug addition device is used to infuse a drug treatment to the live cancer cells. An optical density device is used to capture images of the microbiology sample. An optical microplate spectrophotometric reader coupled to the optical density device is used to measure the density of living cells after infusion of the at least one drug treatment. A computer coupled to the optical microplate spectrophotometric reader is used to determine measured rates of death over a period of time based on the interval population growth data. An optical density application operating on the computer is used process measured rates of death and known predetermined genetic markers, resistances and allergies of the patient associated with the drug to generate clinician treatment recommendations for the patient.

In another embodiment, the drug addition device is used to add a combination of drug treatments to the purified microbiology sample live cancer cells. The optical density device is used to capture optical photometric images of the microbiology sample live cancer cells at different predetermined intervals. At a first interval the image captured is before the addition of drug treatments to establish a baseline growth rate. Additional images are captured at subsequent intervals after infusion of the at least one drug treatment. The subsequent interval captured images are used to measure a change in cell density caused by the drug treatments.

The changes in cell density measurements are used in a process to determine a death rate of the microbiology sample live cancer cells caused by the treatment drugs. The death rate determination is processed with a computer coupled to the optical density device. The interval captured images are recorded and measured to determine the population of the microbiology sample live cancer cells at the different predetermined intervals. In one embodiment at least one computer having an optical density application wirelessly coupled to the computer is used to measure the changes in the microbiology sample population of living cells using the captured images to determine measured rates of death of the living cells over a period of time.

### BRIEF DESCRIPTION OF THE DRAWINGS

FIG. 1 shows for illustrative purposes only an example of an optical density measuring and testing device system of one embodiment.

FIG. 2 shows for illustrative purposes only an example of optical density measurement and testing device results of one embodiment.

FIG. 3 shows for illustrative purposes only an example of an overview of a method and devices for direct apoptosis assay of purified cells of one embodiment.

FIG. 4 shows a block diagram of an overview of a cancer companion diagnostic for chemotherapy of one embodiment.

FIG. 5 shows a block diagram of an overview flow chart of performing a cancer companion diagnostic direct apoptosis assay of purified cancer cells of one embodiment.

FIG. 6 shows a block diagram of an overview flow chart of receiving patient biopsy tissue samples of one embodiment.

FIG. 7 shows a block diagram of an overview flow chart of assaying apoptosis of purified cancer cells in the culture of one embodiment.

FIG. 8 shows a block diagram of an overview flow chart of a direct APOP assay of purified cells of one embodiment.

### DETAILED DESCRIPTION OF THE INVENTION

In the following description, reference is made to the accompanying drawings, which form a part hereof, and which are shown by way of illustration a specific example in which the invention may be practiced. It is to be understood that other embodiments may be utilized, and structural changes may be made without departing from the scope of the present invention.

### General Overview:

The embodiments disclose an optical density measurement and testing system including a purification device configured to detect and isolate live cancer cells of a patient microbiology sample. A drug addition device is used to infuse a drug treatment to the live cancer cells. An optical density device is used to capture images of the microbiology sample. An optical microplate spectrophotometric reader coupled to the optical density device is used to measure the density of living cells after infusion of the at least one drug treatment. A computer coupled to the optical microplate spectrophotometric reader is used to determine measured rates of death over a period of time based on the interval population growth data. An optical density application operating on the computer is used process measured rates of death and known predetermined genetic markers, resistances and allergies of the patient associated with the drug to generate clinician treatment recommendations for the patient.

For the optical density measuring and testing device of the present invention, it should be noted that the descriptions that follow, for example, the term APOP is used to describe apoptosis and related direct APOP assays of purified cells. The descriptions that follow referring to APOP and APOP assays are for illustrative purposes and the underlying system can apply to any number and multiple types of medical drug treatments and systems. In one embodiment of the present invention, the systems and devices used for direct APOP assay of purified cells can be configured using several drugs for testing. The devices for direct APOP assay of purified cells may be configured to include several cell purification technologies and may be configured to include several next-generation sequencing technologies using the present invention.

The term “apoptosis” used herein refers to a genetically directed process of cell self-destruction that is marked by the fragmentation of nuclear DNA, is activated either by the presence of a stimulus or removal of a suppressing agent or stimulus, is a normal physiological process eliminating DNA-damaged, superfluous, or unwanted cells, and when halted (as by genetic mutation) may result in uncontrolled cell growth and tumor formation and additionally is expressed without any change in meaning as “APOP” in any case lower, upper or mixed.

The optical density device includes an optical microplate spectrophotometric reader used to measure the density of living cells.

The term “O.D.” used herein refers to the term “Optical Density” is expressed without any change in meaning as “optical density” in any case lower, upper or mixed.

The term “APOP” used herein refers to an assay to test and measure the apoptosis effectiveness of a single drug or combination of drugs against purified cells including cancer cells.

The term “companion diagnostic” used herein refers to a diagnostic test used as a companion to a therapeutic drug to determine its applicability to a specific person.

The term “antigen” used herein refers to a toxin or other foreign substance which induces an immune response in the body, especially the production of antibodies or a cellular response.

The term “Immunotherapy” used herein refers to a treatment to stimulate or restore the ability of the immune (defense) system to fight infection and disease.

The term “cannabinoid” used herein refers to any chemical in marijuana that causes drug-like effects all through the body, including the central nervous system and the immune system.

The term “CBD” used herein refers to legal nonintoxicating cannabinoids found in cannabis and hemp.

### DETAILED DESCRIPTION

### Optical Density Measuring and Testing Device System:

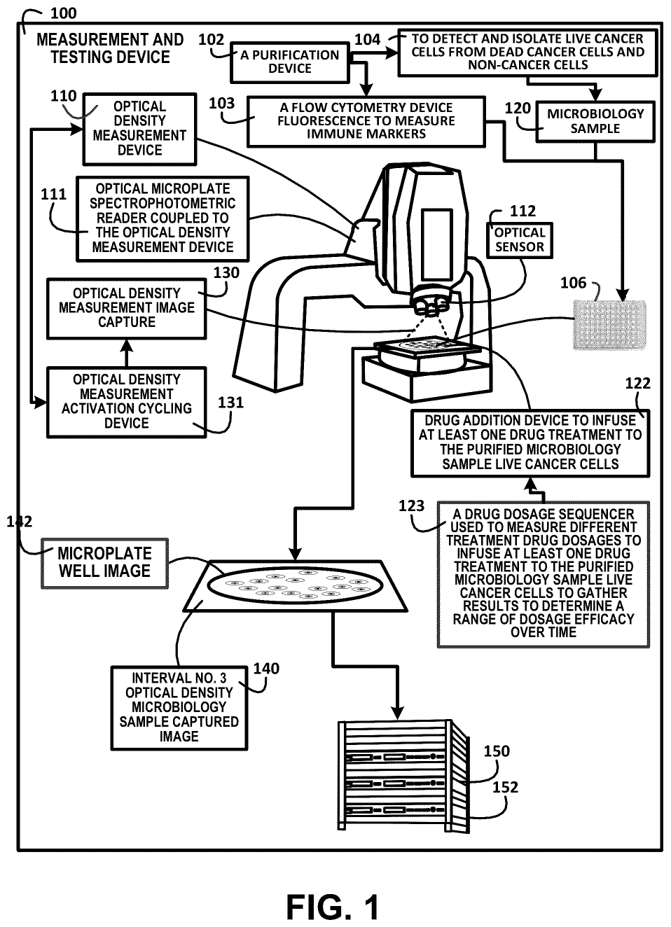

FIG. 1 shows for illustrative purposes only an example of an optical density measuring and testing device system of one embodiment. FIG. 1 shows a purification device 102 that is used to detect and isolate live cancer cells from dead cancer cells and non-cancer cells of a microbiology sample 120. At least one microplate 106 coupled to the purification device 102 is used to contain the microbiology sample 120 of purified microbiology living cells in a liquid. A microplate 106 is a plurality of “wells” to contain cell cultures within a well, which is described as a small test tube. A drug addition device 122 coupled to the at least one microplate 106 is used to infuse at least one drug treatment into the live cancer cells. A drug dosage sequencer device 123 coupled to the drug addition device 122 is used to measure different treatment drug dosages to infuse at least one drug treatment to the purified microbiology sample live cancer cells to gather results to determine a range of dosage efficacy over time.

A measurement and testing device 100 including an optical density measurement device 110. In one embodiment the optical density measurement device 110 is used to determine the concentration of microbiology cells in a liquid culture of a microbiology sample 120. The optical density measurement device 110 captures an image of the microbiology sample 120 for an optical density measurement process of the image captured 130. The optical density measurement device 110 captures a high-resolution image using an optical sensor 112 coupled to the optical density measurement device 110. In this example, an interval no. 3 optical density microbiology sample captured image 140 is stored in a server 150. The server 150 provides the interval no. 3 optical density microbiology sample captured image 140 to a computer to measure the concentration of microbiology cells in the determined area of the microplate 106 well containing the microbiology sample 120. The concentration is determined by the microbiology cell count of individual cells identified by the optical sensor 112. The physical area of the microplate 106 well is a predetermined area.

The results are represented as an average number of microbiology cells in a predetermined area, for example, a square inch and square centimeter. This result is a base concentration at the time the optical density measurement image capture 130 is captured. The concentration determination of the optical density measurement changes and analytical evaluations and is the basis for suggestion decisions for a clinician to consider a treatment plan of one embodiment.

### Optical Density Measurement and Testing Device Results:

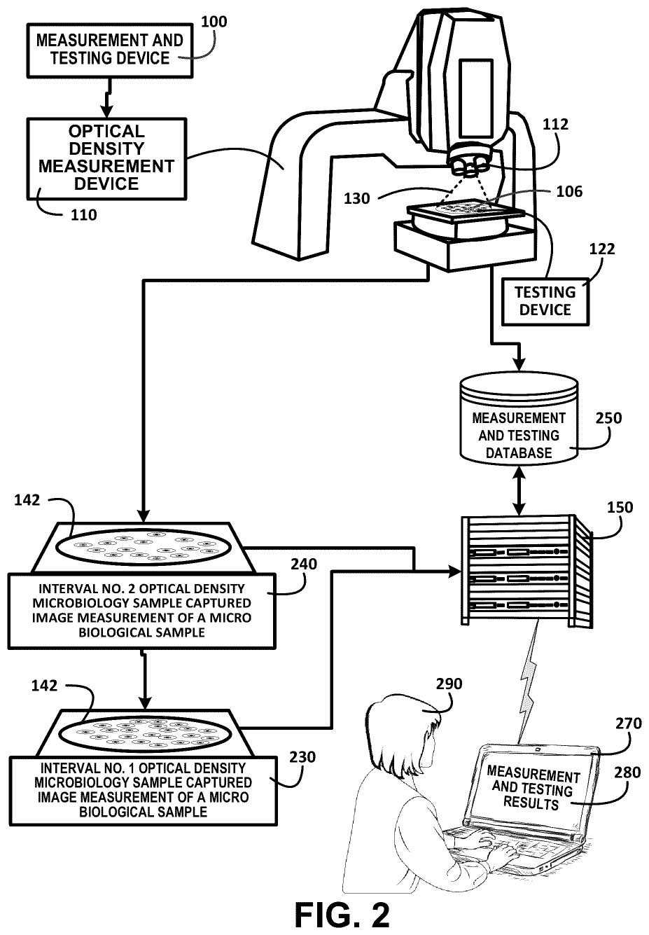

FIG. 2 shows for illustrative purposes only an example of optical density measurement and testing device results of one embodiment. FIG. 2 shows the measurement and testing devices 100 including the optical density measurement device 110, wherein the optical density measurement device 110 can include an optical microplate spectrophotometric reader. The optical density measurement device 110 is used for optical density measurement image captures 130 using a spectrophotometric device optical sensor 112 to capture images of at least one microbiology sample 120 in the microplate 106 of FIG. 1. The optical density of the at least one microbiology sample 120 is measured using a computer 270.

The changes in cell density measurements are used in a process to determine a death rate of the microbiology sample live cancer cells caused by the treatment drugs. The death rate determination is processed with a computer 270 coupled to the server 260 to access the microplate well image 142 stored in the server to perform the concentration determination. The interval captured images are recorded and measured to determine the population of the microbiology sample live cancer cells at the different predetermined intervals. In one embodiment at least one computer having an optical density application wirelessly coupled to the server is used to measure the changes in the microbiology sample population of living cells using the captured images to determine measured rates of death of the living cells over a period of time. The determination results of the measured rates of death of the living cells over a period of time are stored in a measurement and testing database 250 coupled to the server 260.

The optical density measurement image capture 130 is activated in a controlled environment conducive to cell growth. An optical density measurement activation cycling device 131 coupled to the optical density device 130 is used to activate the optical density image capture process at predetermined intervals. The optical density measurement image capture 130 is activated at predetermined time intervals to measure changes in the population of the microbiology cells for a determination of the concentration level. The measurement activation cycle is first performed using the optical density measurement device 110 to measure microbiology cell growth without any treatment to measure a baseline growth rate.

The drug addition device 122 is used for testing of specific treatment drugs added to separate individual microplate cultures of the microbiology sample 120. The treated microbiology samples are tested using the optical density measurement device 110 to determine whether the treatment causes apoptosis of the microbiology samples. A drug dosage sequencer device 123 coupled to the drug addition device is used to measure different treatment drug dosages to infuse at least one drug treatment to the purified microbiology sample live cancer cells to gather results to determine a range of dosage efficacy over time.

The measurement results of the optical density measurement device 110 captured images analysis provide the concentrations of both living microbiology cells and dead microbiology cells, which indicate the efficacy of the treatment over the predetermined time intervals. The optical density measurement device 110 measurement analysis results include the percentages of microbiology cell deaths, the rates at which the population declines (if at all), what time is needed for the treatment to become effective, and how long the treatment effectiveness lasts in terms of time. In these examples, the optical density measurement device 110 measurement activation cycles can be repeated with varying dosages to gather results to determine a range of dosage efficacy over time.

Optical density measurement results of microbiology sample images captured at different intervals are stored in a measurement and testing database 250. FIG. 2 shows an interval no. 1 optical density microbiology sample captured image of a micro biological sample and an interval no. 2 optical density microbiology sample captured image of a micro biological sample. The interval no. 1 captured image is measured to determine a live cell population. The interval no. 2 captured image is measured to determine any change in the live cell population. In this example, the interval no. 2 measured population of live cells shows a decrease over the population measured in the interval no. 1 measured population of live cells cell count. The optical density measurement results are stored in a measurement and testing database 250. A server 260 coupled to the measurement and testing database 250 performs a comparative analysis of changes of the comparative analysis results and sends the comparative analysis results to a computer 270 to display measurement and testing results 280 to a clinician 290 for review on the treatment testing of one embodiment.

### Direct Apoptosis Assay of Purified Cells:

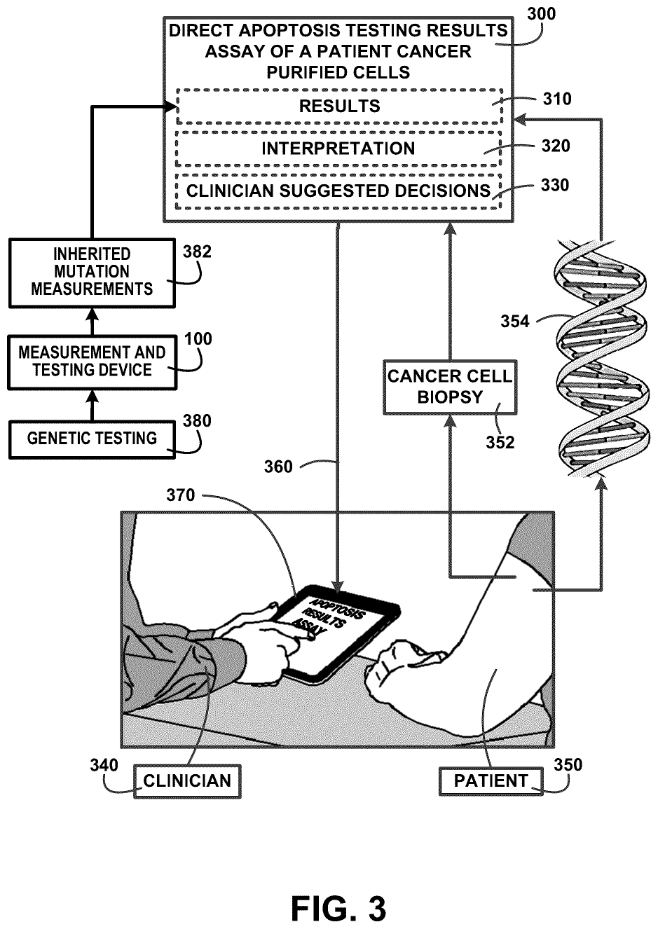

FIG. 3 shows for illustrative purposes only an example of an overview of a method and devices for direct apoptosis assay of purified cells of one embodiment. FIG. 3 shows a patient 350 providing a cancer cell biopsy 352 and DNA genomic testing 354. The method and devices for direct apoptosis assay of purified cells process the cancer cell biopsy 352 and DNA genomic testing 354 provided by the patient 350. The cancer cell biopsy 352 tissues are processed in at least one cell purification procedure. The purified cells then are processed in a series of apoptosis next-generation sequence testing with selected drugs and combinations of drugs to determine which is the most effective in killing in this example the patient's cancer cells. Recommending part inhibitors as part of suggestions to doctors includes using the APOP assay with and/or without next-generation sequencing, oral swabs, and/or b mood in parallel to be able to assess where DNA mutations exist, for example in a tumor or also due to bloodline mutations.

DNA genomic testing 354 is reviewed to identify genetic markers that show any variants in the genes that would affect the use of one or more drugs that could be used in a treatment regimen. Direct apoptosis testing results assay of a patient cancer purified cells 300 are correlated into results 310, interpretations 320, and clinician suggested decisions 330. The results include measurement and testing devices 100 used in genetic testing 380 to measure the genome for inherited mutation measurements 382. The correlated apoptosis testing results assay including the results 310, interpretations 320, and clinician-suggested decisions 330 are transmitted for example to a clinician digital tablet 360. The clinician digital tablet 360 displays the apoptosis testing results assay using an apoptosis application installed 370 on the clinician digital tablet 360. This allows clinician 340 to review the results, interpretations, and suggested decisions with the patient 350 for planning a treatment course of one embodiment.

### Cancer Companion Diagnostic for Chemotherapy:

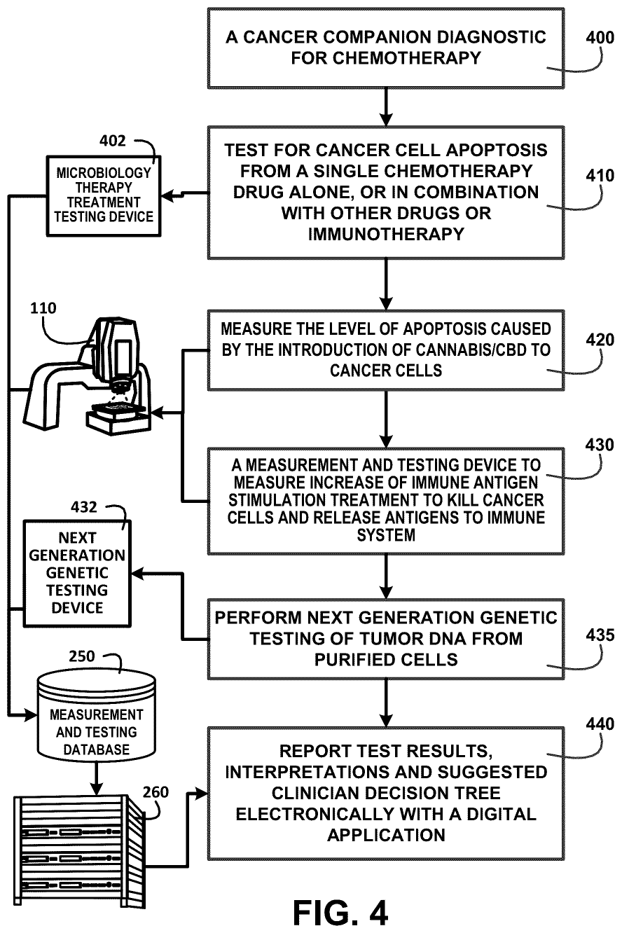

FIG. 4 shows a block diagram of an overview of a cancer companion diagnostic for chemotherapy of one embodiment. FIG. 4 shows a cancer companion diagnostic for chemotherapy 400 used to test for cancer cell apoptosis from a single chemotherapy drug alone, or in combination with other drugs or immunotherapy 410. The companion diagnostic data are stored in the measurement and testing database 250. The direct apoptosis testing results assay of a patient cancer purified cells 300 of FIG. 3 in one sequencing example of the results record a measure of the level of apoptosis caused by the introduction of cannabinoids/CBD to cancer cells 420 and store data in the measurement and testing database 250 for processing in the server 260. Also, measure the increase of immune antigen stimulation treatment to kill cancer cells and release antigens to the immune system 430 and store data in the measurement and testing database 250 for processing in the server 260. Perform next-generation genetic testing of tumor DNA from purified cells 435 and store data in the measurement and testing database 250 for processing in server 260. The direct apoptosis testing results assay of a patient cancer purified cells 300 of FIG. 3 is used to report test results, interpretations, and suggested clinician decision trees electronically with a digital application 440 to make the data available to clinicians for reviewing with patients from the server 260 of one embodiment.

### Performing a Cancer Companion Diagnostic Direct Apoptosis Assay of Purified Cancer Cells:

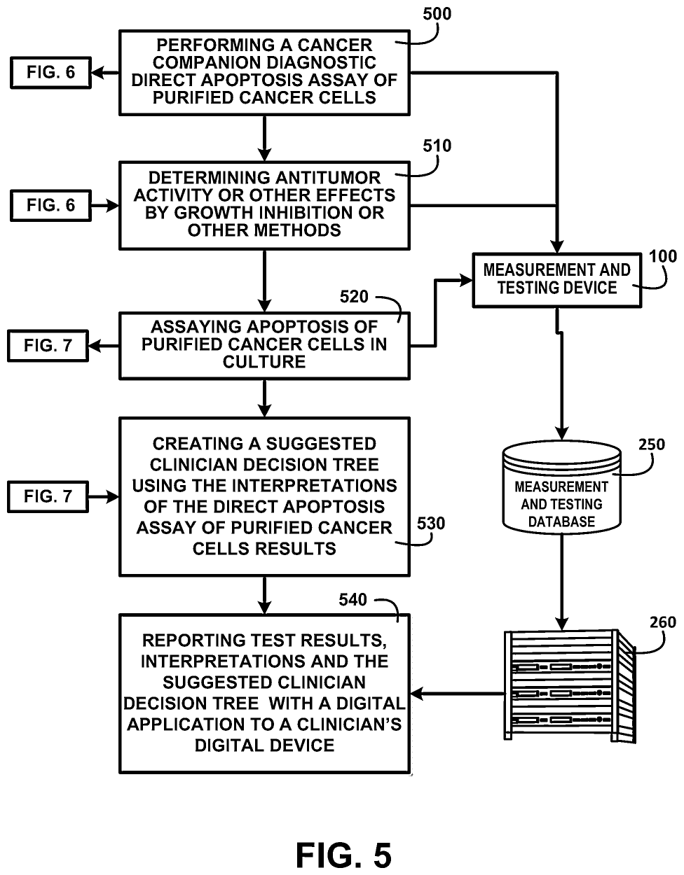

FIG. 5 shows a block diagram of an overview flow chart of performing a cancer companion diagnostic direct apoptosis assay of purified cancer cells of one embodiment. FIG. 5 shows performing a cancer companion diagnostic direct apoptosis assay of purified cancer cells 500 with descriptions of processes shown in FIG. 5. After performing a cancer companion diagnostic direct apoptosis assay of purified cancer cells 500 as shown in FIG. 5 are processes for determining antitumor activity or other effects by growth inhibition or other methods 510. Processing continues with assaying apoptosis of purified cancer cells in culture 520 with descriptions of processes shown in FIG. 5. After performing the processes for assaying apoptosis of purified cancer cells in culture 520 as shown in FIG. 5 the processing continues for creating a suggested clinician decision tree using the interpretations of the direct apoptosis assay of purified cancer cells results 530. The processes include taking measurements using the measurement and testing devices 100 and recording the results in the measurement and testing database 250. A portion of the processes are performed in server 260 including communicating the results to clinicians and specific patients.

The cancer companion diagnostic direct apoptosis assay of purified cancer cells includes reporting test results, interpretations, and the suggested clinician decision tree with a digital application to a clinician's digital device 340 for allowing a clinician and patient to discuss a course of treatment based on the results of the testing for that specific patient of one embodiment.

### Receiving Patient Biopsy Tissue Sample:

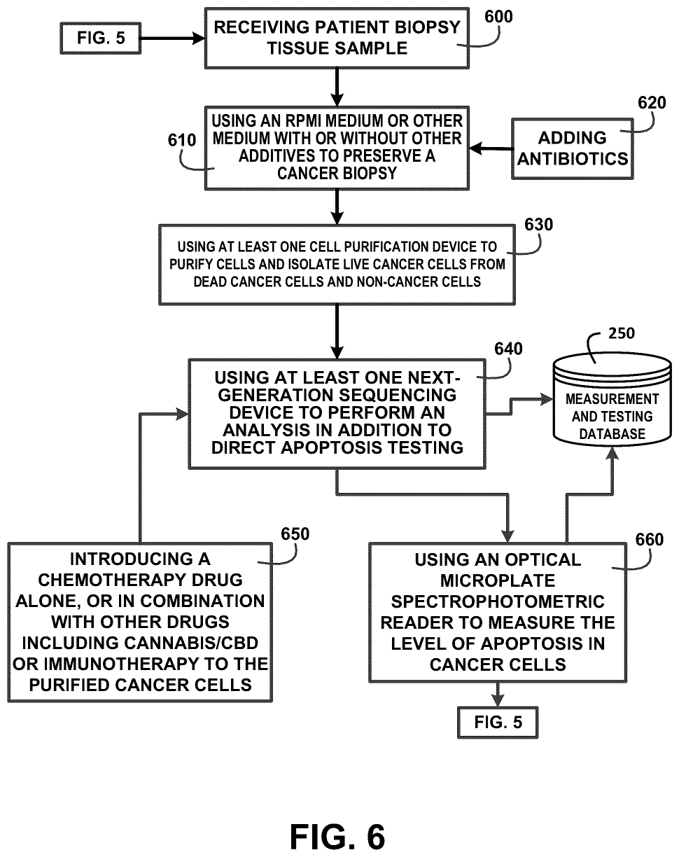

FIG. 6 shows a block diagram of an overview flow chart of receiving patient biopsy tissue samples of one embodiment. FIG. 6 shows processes for the cancer companion diagnostic direct apoptosis assay of purified cancer cells 500 of FIG. 5 that include receiving patient biopsy tissue sample 600. The process includes using an RPMI medium or other medium with or without other additives to preserve a cancer biopsy 610. The process includes adding antibiotics 620 to a portion of the preserved cancel biopsy. Preparation of the cancer cells for testing includes using at least one cell purification device to purify cells and sort out cancer cells 630. Individual tests on the cancer cells are performed using at least one next-generation sequencing device to perform an analysis in addition to direct apoptosis testing 640. The testing data and results are recorded in the measurement and testing database 250.

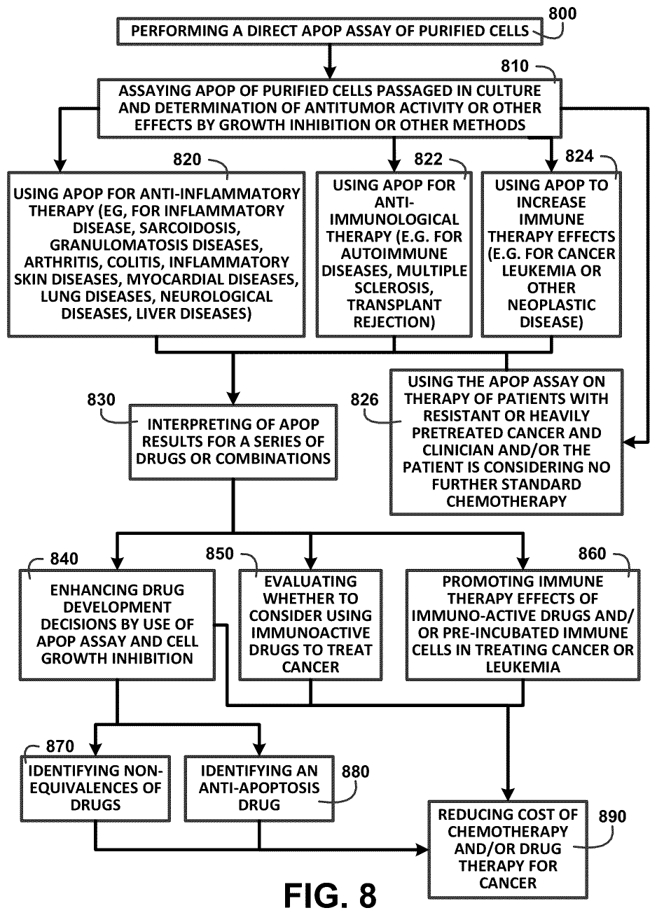

Direct apoptosis testing includes introducing a chemotherapy drug alone, or in combination with other drugs including cannabinoids/CBD or immunotherapy to the purified cancer cells 650. The apoptosis effect of the chemotherapy drug alone, or in combination with other drugs including cannabinoids/CBD or immunotherapy on the purified cancer cells is determined using an optical microplate spectrophotometric reader to measure the level of apoptosis in cancer cells 660. After the determinations of the apoptosis affects the processing returns to FIG. 8 of one embodiment.

### Assaying Apoptosis of Purified Cancer Cells in Culture:

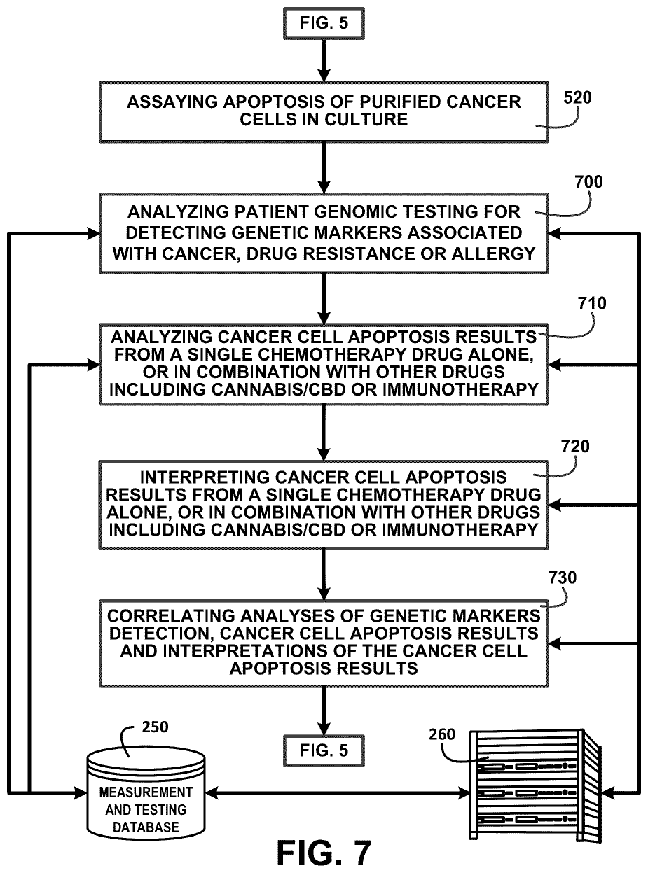

FIG. 7 shows a block diagram of an overview flow chart of assaying apoptosis of purified cancer cells in the culture of one embodiment. FIG. 7 shows a continuation of processing from FIG. 6 that includes assaying apoptosis of purified cancer cells in culture 720. The process includes patient genomic testing using cells from the preserved cancer biopsy and may include analysis of patient blood samples. The effectiveness of various treatments may vary depending on the patient's genetic makeup. The assaying apoptosis processing may include analyzing patient genomic testing for detecting genetic markers associated with cancer, drug resistance, or allergy 700 and in parallel to be able to assess where DNA mutations exist, for example in a tumor or also due to bloodline mutations. The genomic testing data is stored in the measurement and testing database 250.

Analyzing cancer cell apoptosis results from a single chemotherapy drug alone, or in combination with other drugs including cannabinoids/CBD or immunotherapy 710 identifies the potential success of a treatment for the single chemotherapy drug alone, or in combination with other drugs including cannabinoids/CBD or immunotherapy. The cancer cell apoptosis analysis results data are stored in the measurement and testing database 250. Interpreting cancer cell apoptosis results from a single chemotherapy drug alone, or in combination with other drugs including cannabinoids/CBD or immunotherapy 720 assists a clinician in evaluating the testing results. Correlating analyses of genetic markers detection, cancer cell apoptosis results and interpretations of the cancer cell apoptosis results 730 is used in the processes following as described in FIG. 8. The data stored in the measurement and testing database 250 is used for processing in the server 260 and the results of the processing are also stored in the measurement and testing database 250 of one embodiment.

### A Method for Direct APOP Assay of Purified Cells:

FIG. 8 shows a block diagram of an overview flow chart of a method for a direct APOP assay of purified cells of one embodiment. FIG. 8 shows a method for direct APOP assay of purified cells including performing a direct APOP assay of purified cells 800. The method for direct APOP assay of purified cells includes performing the assays on patient-purified cells to assess the effectiveness of drug treatments specific to that patient's current condition including genetics and prior treatment effects. Performing a direct APOP assay of purified cells 800 includes assaying apoptosis of purified cells passaged in culture and determination of antitumor activity or other effects by growth inhibition or other methods 810. Using APOP for anti-inflammatory therapy (e.g., for inflammatory disease, sarcoidosis, granulomatosis diseases, arthritis, colitis, inflammatory skin diseases, myocardial diseases, lung diseases, neurological diseases, liver diseases) 820. Using APOP for anti-immunological therapy (e.g. for autoimmune diseases, multiple sclerosis, transplant rejection) 822. Using APOP to increase immune therapy effects (e.g. for cancer, leukemia, or other neoplastic disease) 824. Using the APOP assay on therapy of patients with resistant or heavily pretreated cancer and clinician and/or the patient is considering no further standard chemotherapy 826.

The method for direct APOP assay of purified cells includes interpreting APOP results for a series of drugs or combinations 830 for suggested clinician decisions in choosing potential treatments. The clinicians may receive the direct APOP assay and suggested clinician decisions using a direct APOP assay of purified cells application installed on a clinician's digital device including a smartphone, digital tablet, and computer. The method for direct APOP assay of purified cells is used for enhancing drug development decisions by use of APOP assay and cell growth inhibition 840, identifying non-equivalences of drugs 870, identifying an anti-Apoptosis drug 880, evaluating whether to consider using immunoactive drugs to treat cancer 850, promoting immune therapy effects of immuno-active drugs and/or immune cells in treating cancer or leukemia 860 and reducing cost of chemotherapy and/or drug therapy for cancer 890 of one embodiment.

### Extended APOP Assay Decision Tree:

The following description shows a block diagram of an overview of the extended APOP assay decision tree of one embodiment. The block diagram shows the extended APOP assay decision tree. The extended APOP assay decision tree includes a correlation of condition, extension, and suggested clinician decision. A condition, includes, for example, APOP assay-cells alone or in combination, an extension, for example, adding immune cells (car-t cells or modified lymphocytes), target cells and measure O.D. and suggested clinician decision, for example, if drugs alone or in combination plus immune cells increase O.D. change >1 S.D., consider adding those drugs or combinations to other immune therapy (e.g. immune cells, checkpoint inhibitors). The extended APOP assay decision tree continues with the same, add immune cells, plus target cells and measure protein release from purified cancer cells, if drugs increase protein release; and consider adding drugs together with immune cells or immuno-oncologic (IO) drugs to increase immune response or consider giving drugs or combinations first and adding immune cells and/or IO drugs later. In the same condition, add target cells with inflammatory cells. If drugs or combinations with added inflammatory cells increase O.D. change >1 S.D. then consider adding drugs or combinations with inflammatory cells. Same condition 1310, if no increase in O.D. change >1 S.D. then, consider not adding the drugs or combinations or inflammatory cells or immune cells of one embodiment.

### Pre-APOP Assay Decision Tree:

The following description shows a block diagram of an overview of the pre-APOP assay decision tree of one embodiment. The pre-APOP assay decision tree using a cell sample. The pre-APOP assay decision tree includes testing and suggested clinician decisions for the series of testing conditions, for example, immunohistology (e.g., estrogen receptor progesterone receptor her2 testing) and if positive, use of hormone blocker or immunological agent.

Additional testing conditions include fish (e.g. her 2 testing), if positive use a biological agent; immune marker testing (e.g. pdl1 or pd1), and if positive use an immunological agent; flow cytometry (to measure targets or markers), and if positive use biological agent; next generation sequencing or hot spot sequencing, and if positive use agent targeted to the mutation, overexpression, or use clinical trial of such a drug. Additional suggested clinician decisions include at the time of progression of cancer leukemia or neoplastic condition, collecting a sample, purifying cells, and performing APOP assay of one embodiment.

### Parallel APOP Assay Decision Tree:

The following description and block diagram shows an overview of the parallel APOP assay decision tree of one embodiment. The parallel APOP assay decision tree with steps that include collecting a cell sample, process, and the parallel test. The parallel APOP assay decision tree correlates the results of APOP, results, and suggested clinician decisions, for example, negative*, wherein * all results of drugs or combinations give an increase in O.D. change≤1.0 S.D., positive, and use drugs but not drugs or combinations from APOP and at progression collect another sample and perform another APOP assay. Another example of positive is wherein a drug or combination produces an increase in O.D. change >1.0 S.D., positive, and use drug from APOP assay.

Blocks are empty and reflect the same results of APOP shown in positive, with positive and/or use drug from APOP first and drug from progression. Continuing with positive, and/or use drug from and drug from APOP at progression. Results of APOP show positive, and negative, and use drugs from APOP and do not use drugs and retest for APOP and at the progression of one embodiment.

### Interpretation of APOP Results for a Series of Drugs or Combinations:

The following description shows a block diagram of an overview of the interpretation of APOP results for a series of drugs or combinations of one embodiment. The block diagram shows step interpretation of APOP results for a series of drugs or combinations. Step interpretation of APOP results for a series of drugs or combinations includes an analysis of multiple drugs and/or combinations and steps to sort drugs and combinations by activity and create a ladder as drugs by the amount of increase in O.D. change. For example, if more than 1 drug or combination produces an increase in O.D. change >1.0 (e.g. drugs A, B, C, but not drugs X, Y, Z) and are within 1 S.D. of each other a suggested clinician decision: includes using the drug/combination A or B or C that has least toxicity or least expense and do not use drug X, Y, or Z, at progression use another A or B or C at progression not previously used or perform another APOP assay and at progression use next most active drug or combinations after A or B or C but not X or Y or Z or perform another APOP assay of one embodiment.

Another example includes only 1 drug or combination that produces the highest change in O.D.>1.0 (e.g. drug F) and by more than 1 S.D. and others do not (e.g. drugs P, Q, R). A suggested clinician decision includes the use of the next most active drug or combination, at progression and the use next most active use drug or combination after F but not P, Q, R, or perform another APOP assay of one embodiment.

Using the APOP Assay on Therapy of Patients with Resistant or Heavily Pretreated Cancer:

The following block diagram shows an overview of using the APOP assay on therapy of patients with resistant or heavily pretreated cancer of one embodiment. The APOP assay on therapy of patients with resistant or heavily pretreated cancer and clinician and/or the patient is considering no further standard chemotherapy. Using the APOP assay on therapy of patients with resistant or heavily pretreated cancer and the clinician and/or the patient is considering no further standard chemotherapy including processing with a tumor biopsy and testing.

### Situations:

The following description and block diagram show an overview of situations of one embodiment. Situations include APOP assay, results, and suggestions for clinician decisions. For example, APOP assay includes all drugs increase in O.D. change≤1.0, negative and consider hospice or supportive/palliative care or clinical trial. APOP assay is the same with a positive result and considers hospice or palliative care, clinical trial, or drugs. The situations continue with CBD or cannabinoid O.D. change >1.0 but chemotherapy drugs all ≤1.0, negative, and consider CBD, cannabinoid, hospice/palliative care, or clinical trial. A drug (e.g. drug X) gives an O.D. change >1.0, negative, consider drug x alone. Same APOP assay, positive, and consider drug X alone or with an additional drug. A drug combination (+/−CBD or cannabinoid gives an O.D. change >1.0 and CBD or cannabinoid O.D. change versus drug or drug combination is <=1.0 S.D.), negative, and consider drug combination alone of one embodiment.

### Situations Continued:

The following description shows a block diagram of an overview of situations continued of one embodiment. The block diagram shows a continuation with situations continued that include the APOP assay, the results, and the suggestion for clinician decision. Examples include same, positive, and consider drug combination alone or with a drug. An APOP assay with drug or combination plus CBD or cannabinoid O.D. change is >1.0 S. D. higher than drug or combination alone, positive, and consider drug or combination with CBD or cannabinoid. Drug or combination plus CBD or cannabinoid O.D. change is >1.0 S. D. higher than drug or combination alone, negative, and consider drug or combination with CBD or cannabinoid but not with a drug of one embodiment.

### Interpretation of APOP Results for Drugs or Combinations Based on the Amount of O.D. Change:

The following description and block diagram show an overview of the interpretation of APOP results for drugs or combinations based on the amount of O.D. change of one embodiment. Interpretation of APOP results for drugs or combinations based on the amount of O.D. changes with an analysis of drug or combination. The analysis of the drug or combination includes APOP change in O.D. and suggested clinician decision. For example, drug (e.g. drug or combination A) results >5 (“very high positive”) and strongly consider using drug A alone or in combination (with hormones, targeted or biological agents or immuneoncology agents or radiation, or surgery).

Drug result >3-5 (“high positive”) with suggested clinician decision, consider using the drug alone or in combination. Drug result >1-3 (“low positive”) and consider using the drug alone or in combination. Drug result ≤1.0 (“negative”) and do not consider using the drug alone but consider other therapy. No drug or combination gives APOP result >1.0 and consider hospice or palliative care, clinical trial, another non-tested drug, or other therapy and consider another biopsy and APOP test of another tumor site. The analysis of drug or combination including APOP assay cannot be performed or is not successful and consider another biopsy and APOP test of another tumor site. Another situation includes at the time of tumor progression considering another biopsy and APOP test of another tumor site of one embodiment.

Interpretation of APOP Results for Drugs with Similar Mechanisms of Action:

The following description shows a block diagram of an overview of the interpretation of APOP results for drugs with similar mechanisms of action of one embodiment. The interpretation of APOP results for drugs with similar mechanisms of action (e.g. “alkylating agents” [cyclophosphamide, ifosfamide, bendamustine] or “platinum” drugs [cisplatin, carboplatin, oxaliplatin] or “tubulin inhibitors” [paclitaxel, docetaxel, nab-paclitaxel]) and includes an analysis of drugs or combinations. The analysis of drugs or combinations is correlated with the result of APOP change in O.D., interpretation, and suggested clinician decision categories. For example, if drug A and drug B O.D. change>1.0 and drug A's O.D. changes>1 S.D. higher than drug B with the interpretation that drug A is superior to drug B 1, and consider using drug A initially, can consider using drug B at progression.

Drug A and drug B O.D. changes >1.0 and O.D. changes are within 1 S.D. of each other, drug A and drug B are equal, and consider using drug A or drug B based on expected toxicity or cost; can consider using another drug B or A at progression. Drug A O.D. change is >1.0 and drug B change is <1.0, drug A is effective and drug B is ineffective, consider using drug A and not using drug B, and at progression consider other therapy or repeat APOP assay. Drug A and drug B O.D. changes are <1.0, neither drug A nor drug B is effective, and consider using other therapy or repeat APOP assay of one embodiment.

Advanced Interpretation of APOP Results Using O.D. Change and Maximum O.D. Increase from a Single Drug or Combination:

The following description shows a block diagram and an overview of the advanced interpretation of APOP results using O.D. change and maximum O.D. increase from a single drug or combination of one embodiment. The step advanced interpretation of APOP results using O.D. change and maximum O.D. increase from a single drug or combination including an analysis of drugs or combinations. The analysis of drugs or combinations a rate of change in O.D., the maximum increase in O.D. units, interpretation of anticellular* effect wherein *anticellular may mean antitumor, anti-leukemia, anti-lymphoid, anti-inflammatory effect, and suggested clinician decision of one embodiment.

The rate of change in O.D. includes, for example, at least four rates of change in O.D. ratings including a high, intermediate, low, and no change. The high, intermediate, and low rates each include a subset of rates for high, intermediate, and low. For example, the rate of change in O.D. high, high, high effect 80; intermediate, high effect 80, and low, high effect 60 with the suggested clinician decision to consider using the drug or combination with the highest anti-cellular effect of one embodiment.

Rate of change in O.D. intermediate, high, high effect 80; intermediate, intermediate effect 60; low, low effect 40 and consider using the drug or combination with the highest anti-cellular effect of one embodiment.

Rate of change in O.D. low, high, low effect 40; intermediate, very low effect 20; low, very low effect 10 and consider using the drug or combination with the highest anti-cellular effect of one embodiment.

Rate of change in O.D. no change, any, no effect drugs inactive and consider using another therapy but not the drugs or combination of one embodiment.

### Enhancing Drug Development Decisions by Use of APOP Assay and Cell Growth Inhibition:

The following description shows a block diagram of an overview of enhancing drug development decisions by use of APOP assay and cell growth inhibition of one embodiment. The enhancing drug development decisions by use of APOP assay and cell growth inhibition with established cancer cell lines plus drug. The enhancing drug development decisions by use of APOP assay and cell growth inhibition combine processes to measure APOP assay O.D. changes and measure inhibition of cell growth. Should the measurements show both tests are negative, then add a drug to other agents in combinations of one embodiment.

When either test is positive, proceed with short-term purified cancer cells in culture. Measure APOP assay O.D. change and measure inhibition of cell growth and if both tests are negative, add the drug together with other agents in combinations. If either test is positive, direct APOP assay of purified cells. If positive results, then suggest a clinical trial of the best drug or drug combination in the diseases from which the purified cells show a positive result and avoid trials in diseases from which purified cells show negative results. If negative results, then add the drug together with other agents of one embodiment.

A Method to Reduce the Cost of Chemotherapy and/or Drug Therapy for Cancer:

The following description shows a block diagram of an overview of a method to reduce the cost of chemotherapy and/or drug therapy for cancer of one embodiment. The block diagram shows a method to reduce the cost of chemotherapy and/or drug therapy for cancer. The method to reduce the cost of chemotherapy and/or drug therapy for cancer includes a cell sample and processing to prepare. The processing to prepare includes cells alone, cells plus expensive single source or multiple single-source drugs, cells plus inexpensive drugs multiple sources or inexpensive generic or single source drugs, cells plus combinations of expensive drugs, cells plus combinations of inexpensive drugs, cells plus inexpensive single drugs+CBD+/−THC, and cells plus inexpensive drug combinations+CBD+/−THC.

The method to reduce the cost of chemotherapy and/or drug therapy for cancer includes a process to identify the most effective therapies and a process to evaluate the cost of the most effective therapies.

The process to evaluate the cost of the most effective therapies is significant as a health plan or hospital or network considers using the least expensive of the most effective therapies, a physician or practice considers using the least expensive of the most effective therapies, the patient considers using the least expensive of the most effective therapies, and state or federal government or governmental agency considers using the least expensive of the most effective therapies of one embodiment.

### Cost of Drugs or Therapies Defined:

The ensuing block diagram shows an overview of the cost of drugs or therapies defined of one embodiment. The cost of drugs or therapies may be defined, as the average sales price, average wholesale price, acquisition price, the net cost to a health plan or network or physician office (after discounts or rebates or other incentives), net cost to the patient, the net cost to the hospital, and patient copay of one embodiment.

A Method to Promote Immune Therapy Effects of Immuno-Active Drugs and/or Immune Cells in Treating Cancer or Leukemia:

The following description is a block diagram of an overview of a method to promote immune therapy effects of immuno-active drugs and/or immune cells in treating cancer or leukemia of one embodiment. The block diagram shows a method to promote the immune therapy effects of immuno-active drugs and/or immune cells in treating cancer or leukemia. The process includes blood samples from a patient with cancer or leukemia. A process to isolate or purify immune cells⁺ where ⁺ immune cells=cells. Processing continues with preincubation with immuno-active drugs (e.g., PD1 or PDL1 or CTLA4 inhibitors alone or in combination with other immuno-active agents) and use as an immune-active cell source. Including a process for immune cells without preincubation with chemotherapy or antineoplastic drug and use as an immuno-active cell source of one embodiment.

### Cancer or Leukemia Cells:

The following description shows a block diagram of an overview of cancer or leukemia cells of one embodiment. The block diagram shows cancer or leukemia cells. The process with cancer or leukemia cells further continues in this description. The process with cancer or leukemia cells includes an APOP assay. The APOP assay includes a process to measure molecule* release into supernatant culture fluid where * molecule >e.g., protein, antigen, cell component. A high release prompts to consider using chemotherapy drugs to increase molecule presentation and immune response including drugs before immunotherapy, drugs together with immunotherapy, and drugs alternating with immunotherapy. A low release and low change in O.D. prompts to consider using immunotherapy alone wherein a progression of cancer leads to repeat APOP assay of one embodiment.

### A Method to Evaluate Whether to Consider Using Immunoactive Drugs to Treat Cancer:

The following description shows a block diagram of an overview of a method to evaluate whether to consider using immunoactive drugs to treat cancer of one embodiment. The block diagram shows a method to evaluate whether to consider using immunoactive drugs to treat cancer. The method to evaluate whether to consider using immunoactive drugs to treat cancer includes APOP assay and if APOP assay change in O.D. is >=1 S.D. higher than, then consider using the immunoactive drugs alone or in combination with other immunoactive agents of one embodiment.

The APOP assay is also performed with chemotherapy or antineoplastic drug, if a change is less than 1 S.D. higher, then consider not using the immunoactive drugs alone or in combination with other immunoactive agents and consider using chemotherapy or antineoplastic drugs alone of one embodiment.

### Measure Immune Marker Before APOP Assay:

The following description shows a block diagram of an overview of measuring immune markers before the APOP assay of one embodiment. The block diagram shows a continuation of cancer or leukemia cells with a process to measure immune markers (e.g., PDL1) before APOP assay. The process includes performing an APOP assay. A process in the APOP assay will measure immune markers in cancer cells remaining after the APOP assay. If there is no increase in immune markers, then consider using chemotherapy only and at progression repeat. If there is an increase in the immune marker, consider chemotherapy and then immunotherapy drug active against the immune marker then proceed to the processes.

If there is an increase in immune markers consider using chemotherapy with an immunotherapy drug active against the immune marker, then proceed to the processes. If there is an increase in immune markers, consider using chemotherapy alternating with immunotherapy drug active against the immune marker then proceed to the processes. If drugs are alleged before testing to be biosimilar or identical but testing with APOP or other tests are found not to be equivalent, then neither drug may be sold as biosimilar or equivalent; this may help extend the marketing of the original drug and force a putative biosimilar to undergo further testing and not be marketed.

### APOP Assay Cancer Cells:

The following description shows a block diagram of an overview of APOP assay cancer cells of one embodiment. The step includes APOP assay with cancer cells alone and APOP assay with cancer cells alone and chemotherapy drugs. The step also includes an APOP assay for cancer cells+preincubated immune cells with O.D. change higher than considering using immune cells preincubated with active drug and considering using the immuno-active drug alone of one embodiment.

An APOP assay of cancer cells+preincubated immune cells where an O.D. change is higher than consider using immune cells preincubated plus chemotherapy or consider using immuno-active drug plus chemotherapy (together, sequential, or alternating) of one embodiment.

An APOP assay of cancer cells+preincubated immune cells with an O.D. change not higher than and is greater than consider not using pre-incubated immune cells and consider not using the immune-active drug alone and consider using chemotherapy alone and at progression consider repeat APOP of one embodiment.

An APOP assay of cancer cells+immune cells not pre-incubated where an O.D. change is higher than, consider using immune cells alone or with chemotherapy if it is high and at progression consider repeat APOP assay of one embodiment.

In the APOP assay cancer cells+immune cells not pre-incubated where an O.D. change is not higher and higher than consider using chemotherapy alone and at progression consider repeat APOP of one embodiment.

### A Method to Identify Non-Equivalences of Drugs:

The following description shows a block diagram of an overview of a method to identify the non-equivalences of drugs of one embodiment. The block diagram shows a method to identify the non-equivalences of drugs. A method to identify non-equivalences of drugs is a process where two or more drugs are compared in the APOP or other assays to determine if they are equivalent or biosimilar. If drugs are alleged before testing to be biosimilar or identical but testing with APOP or other tests are found not to be equivalent, then neither drug may be sold as biosimilar or equivalent; this may help extend the marketing of the original drug and force a putative biosimilar to undergo further testing and not be marketed. This may identify other comparable drugs that may have equal or greater effectiveness and may be able to reduce cost with their use of one embodiment.

### Using the APOP Assay:

The following description shows a block diagram of an overview of using the APOP assay of one embodiment. Using the APOP assay where cancer cells are purified (from cancer patients or long-term cancer cell lines or from cancer patients' short-term cell lines). Cells are tested in the APOP assay with 2 or more drugs (e.g., drug A which may be proprietary and drug B which may be the same structural or biosimilar drug that is generic of one embodiment.

The testing includes cells alone with O.D.; cells+drug A with O.D.; cells+drug B with O.D.; cells with another drug known to produce Apoptosis+drug A with O.D.; and cells with another drug known to produce Apoptosis+drug B with O.D. If the drugs differ more than a defined amount (e.g., 1 S.D.) then the drugs are not equivalent. If one drug differs from another by more than a defined amount (e.g., 1 S.D.) then the drugs are not equivalent of one embodiment.

Cancer cells are purified (from cancer patients, from long-term cancer cell lines, from cancer patients' short-term cell lines) then cells are tested in culture for inhibition of growth rate in vitro. Testing results reach the same conclusions, if differs by more than a defined amount (e.g., 1 S.D.) then the drugs are not equivalent and if differs by more than a defined amount (e.g., 1 S.D.) then the drugs are not equivalent of one embodiment.

### A Method for Identifying an Anti-Apoptosis Drug:

The following description shows a block diagram of an overview of a method for identifying an anti-Apoptosis drug of one embodiment. The block diagram shows a method for identifying an anti-Apoptosis drug. This determines if a drug decreases, inhibits, delays, or prevents Apoptosis (e.g., to prevent or delay Alzheimer's disease, Parkinson's disease, aging, degenerative disease, cancer, Neoplastic disease, or others).

A method for identifying an anti-Apoptosis drug uses long-term cell lines or cells from a patient or short-term cell line from a patient and perform an APOP assay with an agent known to produce Apoptosis with or without a drug to be tested (e.g., drug X). The APOP assay with an agent known to produce Apoptosis with or without a drug to be tested (e.g., drug X) includes cells alone with O.D.; cells+Apoptosis-inducing agent with O.D.; cells+Apoptosis-inducing agent+drug X with O.D.; and cells+drug X with O.D. If is less than by some amount (e.g., over 1 S.D.) then drug X is an anti-Apoptosis drug of one embodiment.

### Direct APOP Assay of Purified Cells Application:

The following description shows for illustrative purposes only an example of a direct APOP assay of purified cells application of one embodiment. The description shows a direct APOP assay of purified cell application used in processing direct APOP assay results. A patient visits a doctor's office/hospital/laboratory to provide a biopsy tissue sample for determination of a diagnosis and treatment plan. The patient's biopsy tissue sample is conveyed for assaying APOP of purified cells. Results of APOP, testing results, and suggested clinician decisions are transmitted to a direct APOP assay network to record, and perform APOP assay, testing results, and suggested clinician decision correlation matrix.

The direct APOP assay network is used for controlling at least one cell purification device for purifying tissue sample cells and for example long-term cancer cell lines. The direct APOP assay network is used for controlling at least one next-generation sequencer device used in performing direct APOP assay of purified cell testing. Receiving and processing tissue samples, processing using at least one cell purification device, and testing using at least one next-generation sequencer device or not includes using at least one sterile enclosure of one embodiment.

The direct APOP assay network includes a plurality of digital servers, a plurality of digital databases, at least one computer, at least one digital processor, at least one communication device with internet connectivity (not shown), at least one communication device with cellular connectivity (not shown) and at least one printer. At least one digital processor correlates the APOP assay, testing results, and suggested clinician decision data into a predetermined format including a matrix. Predetermined formats include electronic and digital formats for transmission to doctor's office/hospital/laboratory using different operating systems and computing languages and display formats. The direct APOP assay of purified cells application is configured in one embodiment to transmit the predetermined formats using internet transmission of direct APOP assay to doctor's office/hospital/laboratory computers. In another embodiment, the direct APOP assay of purified cells application is configured for communicating and transmitting over cellular smartphone communication with a cellular tower to doctor's digital devices with direct APOP assay of purified cells application. Doctor's digital devices including a smart cell phone, a digital tablet, and a laptop computer may each have a different operating system. The direct APOP assay of purified cells application is configured to operate with various operating systems of one embodiment.

The foregoing has described the principles, embodiments, and modes of operation of the present invention. However, the invention should not be construed as being limited to the embodiments discussed. The above-described embodiments should be regarded as illustrative rather than restrictive, and it should be appreciated that variations may be made in those embodiments by workers skilled in the art without departing from the scope of the present invention as defined by the following claims.

1. An optical density measurement and testing system, comprising:
a purification device configured to detect and isolate live cancer cells from dead cancer cells and non-cancer cells using at least one cell isolation system on a microbiology sample of a patient;
a drug addition device configured to infuse at least one drug treatment to the live cancer cells using a drug dosage sequencer device to infuse a predetermined drug dosage of the at least one drug treatment into the live cancer cells on at least one microplate;
an optical density measurement device configured to capture images of the microbiology sample and measure a number, fragments and a diameter of the live cancer cells after the at least one drug treatment with an optical microplate spectrophotometric reader;
wherein the optical microplate spectrophotometric reader coupled to the optical density measurement device is configured to measure the density of living cells to detect and record a population growth of the microbiology sample live cancer cells at different predetermined intervals after infusion of the at least one drug treatment;
a computer coupled to the optical microplate spectrophotometric reader programmed to determine measured rates of death over a period of time based on the population growth of the microbiology sample live cancer cells at the different predetermined intervals;
an optical density application operating on the computer programmed to use the measured rates of death and known predetermined genetic markers, resistances and allergies of the patient associated with the drug to generate clinician treatment recommendations for the patient; and
a molecule release measurement device remotely coupled to the optical microplate configured to measure drug antitumor cell death activity using a level of immune antigen stimulation.

2. The optical density measurement and testing system of claim 1, further comprising at least one microplate coupled to the purification device configured to contain the microbiology sample of purified microbiology living cells in a liquid.

3. The optical density measurement and testing system of claim 1, further comprising an optical sensor coupled to the optical density measurement device configured to capture an image of the microbiology sample of purified microbiology living cells in a liquid contained in the at least one microplate.

4. The optical density measurement and testing system of claim 1, wherein the measured rates of death include at least four rates of change including high, intermediate, low, and no change.

5. The optical density measurement and testing system of claim 1, further comprising a database coupled to a server to store the captured images of the microbiology samples.

6. The optical density measurement and testing system of claim 1, wherein molecule release measurement device configured to compare levels of an immune antigen release rate of cells using different treatments to determine drug treatment effectiveness including drugs together with immunotherapy.

7. The optical density measurement and testing system of claim 1, wherein the at least one computer is programmed having the optical density application configured to determine the measured rates of death results include percentages of microbiology cell deaths, the rates at which the population declines, if at all, what time is needed for the treatment to become effective, and how long the treatment effectiveness lasts in terms of time.

8. An optical density measurement and testing system, comprising:
a purification device configured to detect and isolate live cancer cells from dead cancer cells and non-cancer cells using at least one cell isolation system on a microbiology sample of a patient;
a drug addition device configured to infuse at least one drug treatment to the live cancer cells using a drug dosage sequencer device to infuse a predetermined drug dosage of the at least one drug treatment into the live cancer cells on at least one microplate;
a drug dosage sequencer device coupled to the drug addition device configured to measure different treatment drug dosages to infuse at least one drug treatment to the purified microbiology sample live cancer cells to gather results to determine a range of dosage efficacy over time;
an optical density measurement device configured to capture images of the microbiology sample and measure a number, fragments and a diameter of the live cancer cells after the at least one drug treatment with an optical microplate spectrophotometric reader;
wherein the optical microplate spectrophotometric reader coupled to the optical density measurement device is configured to measure the density of living cells to detect and record a population growth of the microbiology sample live cancer cells at different predetermined intervals after infusion of the at least one drug treatment;
a computer coupled to the optical microplate spectrophotometric reader programmed to determine measured rates of death over a period of time based on the population growth of the microbiology sample live cancer cells at the different predetermined intervals;
an optical density application operating on the computer programmed to use the measured rates of death and known predetermined genetic markers, resistances and allergies of the patient associated with the drug to generate clinician treatment recommendations for the patient;
a molecule release measurement device remotely coupled to the optical microplate configured to measure drug antitumor cell death activity using a level of immune antigen stimulation; and
an immune marker testing system coupled to the microplate configured to identify configured to correlate effectiveness of adding immune cells with other treatment drugs to the live cancer cells.

9. The optical density measurement and testing system of claim 8, further comprising at least one microplate coupled to the purification device configured to contain the microbiology sample of purified microbiology living cells in a liquid.

10. The optical density measurement and testing system of claim 8, wherein the measured rates of death include at least four rates of change including high, intermediate, low, and no change.

11. The optical density measurement and testing system of claim 8, wherein the drug addition device is further configured to infuse the at least one treatment drug alone or in combination with additional treatment drugs into separate cultures of the purified microbiology sample live cancer cells in at least one microplate.

12. The optical density measurement and testing system of claim 8, wherein at least one computer programmed having the optical density application configured to determine the measured rates of death results include the percentages of microbiology cell deaths, the rates at which the population declines, if at all, what time is needed for the treatment to become effective, and how long the treatment effectiveness lasts in terms of time.

13. The optical density measurement and testing system of claim 8, further comprising an optical density measurement activation cycling device coupled to the optical density measurement device configured to activate an optical density image capture process at the predetermined intervals to capture multiple images for measurement using the optical density device.

14. The optical density measurement and testing system of claim 8, further comprising a first predetermined interval of a measurement activation cycle is first performed using an optical density measurement device to measure microbiology cell population growth without any treatment drug additions to measure a baseline growth rate.

15. An optical density measurement and testing system, comprising:
a purification device configured to detect and isolate live cancer cells from dead cancer cells and non-cancer cells using at least one cell isolation system on a microbiology sample of a patient;
a drug addition device configured to infuse at least one drug treatment to the live cancer cells using a drug dosage sequencer device to infuse a predetermined drug dosage of the at least one drug treatment into the live cancer cells on at least one microplate;
an optical density measurement device configured to capture images of the microbiology sample and measure a number, fragments and a diameter of the live cancer cells after the at least one drug treatment with an optical microplate spectrophotometric reader;
wherein the optical microplate spectrophotometric reader coupled to the optical density measurement device is configured to measure the density of living cells to detect and record a population growth of the microbiology sample live cancer cells at different predetermined intervals after infusion of the at least one drug treatment;
an optical density measurement activation cycling device coupled to the optical density device configured to activate the optical density image capture process at the predetermined intervals;
a computer coupled to the optical microplate spectrophotometric reader programmed to determine measured rates of death over a period of time based on the population growth of the microbiology sample live cancer cells at the different predetermined intervals;
an optical density application operating on the computer programmed to use the measured rates of death and known predetermined genetic markers, resistances and allergies of the patient associated with the drug to generate clinician treatment recommendations for the patient;
a molecule release measurement device remotely coupled to the optical microplate configured to measure drug antitumor cell death activity using a level of immune antigen stimulation;
an immune marker testing system coupled to the microplate configured to identify configured to correlate effectiveness of adding immune cells with other treatment drugs to the live cancer cells; and
an anti-apoptosis drug identification system coupled to the microplate configured to identify a drug treatment effectiveness for predetermined conditions.

16. The optical density measurement and testing system of claim 15, wherein identified anti-apoptosis drugs used to treat at least one predetermined condition for effectiveness of apoptosis of cells related to the predetermined condition.

17. The optical density measurement and testing system of claim 15, further comprising a drug dosage sequencer device coupled to the drug addition device is configured to measure using an optical microplate spectrophotometric reader different treatment drug dosages apoptosis results to infuse the at least one drug treatment to the purified microbiology sample live cancer cells to gather results to determine a range of dosage efficacy over time.

18. The optical density measurement and testing system of claim 15, wherein at least one computer having the optical density application programmed to determine the measured rates of death results include the percentages of microbiology cell deaths, the rates at which a population declines, if at all, what time is needed for the treatment to become effective, and how long the treatment effectiveness lasts in terms of time.

19. The optical density measurement and testing system of claim 15, wherein the drug addition device is further configured to infuse the at least one treatment drug alone or in combination with additional treatment drugs into separate cultures of the purified microbiology sample live cancer cells.

20. The optical density measurement and testing system of claim 15, further comprising an optical sensor coupled to the optical density measurement device configured to capture an image of the microbiology sample of purified microbiology living cells in a liquid contained in the at least one microplate.
